(19) United States
(12) Patent Application Publication
(10) Pub. No.: US 2025/0295685 A1
(43) Pub. Date: Sep. 25, 2025

(54) COMPOSITION FORMULATION WITH NMN ANTI-AGING EFFECT AND PREPARATION METHOD THEREOF

(71) Applicant: NANO BIOLOGY LIMITED, RENTON, WA (US)

(72) Inventors: Xinxin LU, Ankang (CN); Likang CHEN, Ankang (CN); Yukang ZHANG, Ankang (CN)

(73) Assignee: NANO BIOLOGY LIMITED, RENTON, WA (US)

(21) Appl. No.: 18/616,008

(22) Filed: Mar. 25, 2024

(51) Int. Cl. A61K 31/706; A61K 9/48; A61K 31/122; A61K 31/197; A61K 31/355; A61K 31/375; A61K 31/4745; A61K 31/704; A61K 38/39

(52) U.S. Cl. CPC A61K 31/706; A61K 9/4833; A61K 31/122; A61K 31/197; A61K 31/355; A61K 31/375; A61K 31/4745; A61K 31/704; A61K 38/39

(57) ABSTRACT

The present invention relates to the technical field of dietary supplements, and more particularly to a composition formulation having an NMN anti-aging effect and a preparation method thereof. Technical Problem: the composition formulation having an NMN anti-aging effect and the preparation method thereof aim to solve the technical problem of not being able to enhance the body's resistance while also having an anti-aging effect, making the patient's body more energetic. Technical Solution: a composition formulation having an NMN anti-aging effect, including the following components: NMN 300 mg, PQQ 10 mg, coenzyme Q10 20 mg, ginsenoside 70 mg, aminobutyric acid 100 mg, other beneficial ingredients q. s.

### TECHNICAL FIELD

The present invention relates to the technical field of dietary supplements, and more particularly to a composition formulation having an NMN anti-aging effect and a preparation method thereof.

### BACKGROUND

NMN, a key intermediate in the synthesis of oxidized coenzyme I (NAD+) widely exists in various organisms in nature, participates in a variety of biochemical reactions in the human body and is closely related to physiological activities such as human immune response, DNA modification, and energy metabolism. NMN, only a small number of users will have, mainly manifested as inflammation in the corners of the mouth, dry mouth, dry stool, and other symptoms. Generally speaking, this manifestation is a phenomenon of excessive body nutrition, so it is urgently needed to provide a composition with NMN anti-aging effect, which is able to enhance the body's resistance while also having an anti-aging effect, making the patient's body more energetic.

### SUMMARY

In view of the deficiencies of the prior art, it is an object of the present invention to provide a composition formulation having an NMN anti-aging effect and a preparation method thereof, wherein the composition formulation having an NMN anti-aging effect and the preparation method thereof, aiming to solve the technical problem of not being able to enhance the body's resistance while also having anti-aging effect, making the patient's body more energetic.

The technical solution of the present invention is as follows: a composition formulation having an NMN anti-aging effect, including the following components: NMN 300 mg, PQQ 10 mg, coenzyme Q10 20 mg, ginsenoside 70 mg, aminobutyric acid 100 mg, other beneficial ingredients q. s.

Preferably, other beneficial ingredients can be chosen, including vitamin C 5 mg-25 mg, vitamin E 2.5 mg-15 mg, and collagen 5 mg-15 mg, in mass percentage: vitamin C 1-5%, vitamin E 0.5-3%, and collagen 1-3%.

Preferably, the mass ratio of NMN, PQQ, and coenzyme Q10 is 30:1:2.

A method for preparing a composition formulation having an NMN anti-aging effect, including the above-mentioned composition formulation having an NMN anti-aging effect, and the steps thereof are as follows:
step 1: crushing:
placing NMN, PQQ, coenzyme Q10, ginsenoside, aminobutyric acid 100 mg, and other ingredients into a crushing device for crushing to ensure that they can be uniformly mixed;
step 2: mixing:
placing the crushed ingredients into a mixing device to ensure uniform distribution and stability;
step 3: mass control:
using precision balance, analytical balance, or other devices to make accurate weighing and mass control;
step 4: preparing capsules:
filling the mixture into capsules using a capsule-filling machine to form capsule granules; and
step 5: packaging:
packaging the prepared composition using a capsule packaging machine.

Preferably, the crushing device in step 1 can be a crusher or a grinder.

Preferably, all the ingredients can be thoroughly mixed using a mechanical stirrer, an ultrasonic stirrer, or other device to ensure uniform distribution and stability.

Preferably, the rotating speed of the mixing device such as the mechanical stirrer or the ultrasonic stirrer in step 2 is 300-500 rpm.

Preferably, in step 1 and step 2, a temperature of 20-25° C. or slightly higher than room temperature should be maintained during mixing and stirring, and a temperature controller or a hot air dryer may be used to maintain a constant mixing temperature.

Preferably, in step 3, a necessary analysis and test can be performed by using instruments such as a spectrophotometer and high-performance liquid chromatography to ensure the quality and efficacy of the product.

Advantageous Effects of the Invention are as follows: NMN, as a key intermediate in the synthesis of NMN anti-aging compositions, participates in regulating cellular energy metabolism, DNA repair, apoptosis, and antioxidant processes and is associated with anti-aging and health maintenance. PQQ has antioxidant activity, which can protect cells from oxidative stress and free radical damage. Studies have shown that PQQ may help improve memory and cognitive function, increase energy metabolism efficiency, and promote cell proliferation. Coenzyme Q10, as an important antioxidant and key substance in cellular energy production, participates in mitochondrial energy metabolism, provides the energy needed by cells, and has antioxidant properties, contributing to protecting cells from free radical damage. Ginsenoside is extracted from ginseng and has the potential effects of anti-fatigue, improving immunity, and improving cognitive function. It is widely used in traditional herbal medicine and is considered to have the effects of promoting physical health and delaying aging. Aminobutyric acid is an amino acid that is a component of protein and participates in a variety of biochemical reactions. It can provide intracellular nutrition and materials for construction, and participate in the synthesis of proteins and other important molecules. Therefore, the composition can increase cellular energy production, promote antioxidant defense, improve immune response, improve cognitive function, reduce fatigue, maintain cellular health, and improve the body resistance of patients while having anti-aging effects, making the patient's body more energetic.

### DETAILED DESCRIPTION OF THE EMBODIMENTS

The present invention will now be further described with reference to the examples.

### Example 1

The present invention provides an example: a composition formulation having an NMN anti-aging effect, including the following components: NMN 300 mg, PQQ 10 mg, coenzyme Q10 20 mg, ginsenoside 70 mg, aminobutyric acid 100 mg, other beneficial ingredients q. s.

Preferably, other beneficial ingredients can be chosen, including vitamin C 5 mg-25 mg, vitamin E 2.5 mg-15 mg, and collagen 5 mg-15 mg, in mass percentage: vitamin C 1-5%, vitamin E 0.5-3%, and collagen 1-3%.

Preferably, the mass ratio of NMN, PQQ, and coenzyme Q10 is 30:1:2.

A method for preparing a composition formulation having an NMN anti-aging effect includes the above-mentioned composition formulation having an NMN anti-aging effect, and the steps thereof are as follows:

### Step 1: Crushing.

To a crushing device, 300 mg of NMN, 10 mg of PQQ, 20 mg of coenzyme Q10, 70 mg of ginsenoside, and 100 mg of aminobutyric acid were added for crushing to ensure that they can be uniformly mixed. The crushing device may be a crusher or a grinder.

### Step 2: Mixing.

The crushed ingredients were fed into a mixing device to ensure uniform distribution and stability. A device such as a mechanical stirrer or an ultrasonic stirrer can be used to sufficiently mix all the ingredients to ensure uniform distribution and stability. The rotation speed was 400 rpm. Since a temperature of 22° C. at room temperature or slightly higher than room temperature should be maintained, a temperature controller or a hot air dryer can be used to maintain a constant mixing temperature.

### Step 3: Mass Control.

Precision balance, analytical balance, or other devices can be used for accurate weighing and quality control. At the same time, a necessary analysis and test can be performed by using instruments such as a spectrophotometer and high-performance liquid chromatography to ensure the quality and efficacy of the product.

### Step 4: Preparing Capsules.

The mixture was filled into capsules using a capsule-filling machine to form capsule granules.

### Step 5: Packaging.

The prepared composition was packaged by using a capsule packaging machine.

### Example 2

The present invention provides an example: a composition formulation having an NMN anti-aging effect includes the following components: NMN 300 mg, PQQ 10 mg, coenzyme Q10 20 mg, ginsenoside 70 mg, aminobutyric acid 100 mg, other beneficial ingredients q. s.

Preferably, other beneficial ingredients can be chosen, including vitamin C 5 mg-25 mg, vitamin E 2.5 mg-15 mg, and collagen 5 mg-15 mg, in mass percentage: vitamin C 1-5%, vitamin E 0.5-3%, and collagen 1-3%.

Preferably, the mass ratio of NMN, PQQ, and coenzyme Q10 is 30:1:2.

A method for preparing a composition formulation having an NMN anti-aging effect includes the above-mentioned composition formulation having an NMN anti-aging effect, and the steps thereof are as follows:

### Step 1: Crushing.

To a crushing device, 300 mg of NMN, 10 mg of PQQ, 20 mg of coenzyme Q10, 70 mg of ginsenoside, 100 mg of aminobutyric acid, 5 mg of vitamin C, 2.5 mg of vitamin E, and 5 mg of collagen protein were added for crushing to ensure that they can be uniformly mixed. The crushing device may be a crusher or a grinder.

### Step 2: Mixing.

The crushed ingredients were fed into a mixing device to ensure uniform distribution and stability. A device such as a mechanical stirrer or an ultrasonic stirrer can be used to sufficiently mix all the ingredients to ensure uniform distribution and stability. The rotation speed was 500 rpm. Since a temperature of 250 at room temperature or slightly higher than room temperature should be maintained, a temperature controller or a hot air dryer can be used to maintain a constant mixing temperature.

### Step 3: Mass Control.

Precision balance, analytical balance, or other devices can be used for accurate weighing and quality control. At the same time, a necessary analysis and test can be performed by using instruments such as a spectrophotometer and high-performance liquid chromatography to ensure the quality and efficacy of the product.

### Step 4: Preparing Capsules.

The mixture was filled into capsules using a capsule-filling machine to form capsule granules.

Step 5: packaging.

The prepared composition was packaged by using a capsule packaging machine.

### Example 3

The present invention provides an example: a composition formulation having an NMN anti-aging effect including the following components: NMN 300 mg, PQQ 10 mg, coenzyme Q10 20 mg, ginsenoside 70 mg, aminobutyric acid 100 mg, other beneficial ingredients q. s.

Preferably, other beneficial ingredients can be chosen, including vitamin C 5 mg-25 mg, vitamin E 2.5 mg-15 mg, and collagen 5 mg-15 mg, in mass percentage: vitamin C 1-5%, vitamin E 0.5-3%, and collagen 1-3%.

Preferably, the mass ratio of NMN, PQQ, and coenzyme Q10 is 30:1:2.

A method for preparing a composition formulation having an NMN anti-aging effect includes the above-mentioned composition formulation having an NMN anti-aging effect, and the steps thereof are as follows:

### Step 1: Crushing.

To a crushing device, 300 mg of NMN, 10 mg of PQQ, 20 mg of coenzyme Q10, 70 mg of ginsenoside, 100 mg of aminobutyric acid, 25 mg of vitamin C, 15 mg of vitamin E, and 15 mg of collagen protein were added for crushing to ensure that they can be uniformly mixed. The crushing device may be a crusher or a grinder.

### Step 2: Mixing.

The crushed ingredients were fed into a mixing device to ensure uniform distribution and stability. A device such as a mechanical stirrer or an ultrasonic stirrer can be used to sufficiently mix all the ingredients to ensure uniform distribution and stability. The rotation speed was 300 rpm. Since a temperature of 200 at room temperature or slightly higher than room temperature should be maintained, a temperature controller or a hot air dryer can be used to maintain a constant mixing temperature.

### Step 3: Mass Control.

Precision balance, analytical balance, or other devices can be used for accurate weighing and quality control. At the same time, a necessary analysis and test can be performed by using instruments such as a spectrophotometer and high-performance liquid chromatography to ensure the quality and efficacy of the product.

### Step 4: Preparing Capsules.

The mixture was filled into capsules using a capsule-filling machine to form capsule granules.

### Step 5: Packaging.

The prepared composition was packaged by using a capsule packaging machine.

What is claimed is:

1. A composition formulation having an NMN anti-aging effect, comprising the following components: NMN 300 mg, PQQ 10 mg, coenzyme Q10 20 mg, ginsenoside 70 mg, aminobutyric acid 100 mg, other beneficial ingredients q. s.

2. The composition formulation having an NMN anti-aging effect according to claim 1, wherein other beneficial ingredients can be chosen, comprising vitamin C 5 mg-25 mg, vitamin E 2.5 mg-15 mg, and collagen 5 mg-15 mg; in mass percentage, vitamin C 1-5%, vitamin E 0.5-3%, and collagen 1-3%.

3. The composition formulation having an NMN anti-aging effect according to claim 1, wherein the mass ratio of NMN, PQQ, and coenzyme Q10 is 30:1:2.

4. A method for preparing a composition formulation having an NMN anti-aging effect, comprising the composition formulation having an NMN anti-aging effect according to claim 1, and the steps thereof are as follows:
step 1: crushing:
placing NMN, PQQ, coenzyme Q10, ginsenoside, aminobutyric acid 100 mg, and other ingredients into a crushing device for crushing to ensure that they can be uniformly mixed;
step 2: mixing:
placing the crushed ingredients into a mixing device to ensure uniform distribution and stability;
step 3: mass control:
using precision balance, analytical balance, or other devices to make accurate weighing and mass control;
step 4: preparing capsules:
filling the mixture into capsules using a capsule-filling machine to form capsule granules; and
step 5: packaging:
packaging the prepared composition using a capsule packaging machine.

5. The method for preparing the composition formulation having an NMN anti-aging effect according to claim 3, wherein the crushing device in step 1 can be a crusher or a grinder.

6. The method for preparing the composition formulation having an NMN anti-aging effect according to claim 3, wherein in step 2, all the ingredients can be thoroughly mixed using a mechanical stirrer, an ultrasonic stirrer, or other devices to ensure uniform distribution and stability.

7. The method for preparing the composition formulation having an NMN anti-aging effect according to claim 6, wherein the rotating speed of the mixing device such as the mechanical stirrer or the ultrasonic stirrer in step 2 is 300-500 rpm.

8. The method for preparing the composition formulation having an NMN anti-aging effect according to claim 3, wherein in step 1 and step 2, a temperature of 20-25° C. or slightly higher than room temperature should be maintained during mixing and stirring, and a temperature controller or a hot air dryer can be used to maintain a constant mixing temperature.

9. The method for preparing the composition formulation having an NMN anti-aging effect according to claim 3, wherein in step 3, a necessary analysis and test can be performed by using instruments such as a spectrophotometer and high-performance liquid chromatography to ensure the quality and efficacy of the product.
